(19) United States
(12) Patent Application Publication
(10) Pub. No.: US 2018/0360453 A1
(43) Pub. Date: Dec. 20, 2018

(54) MESH SUTURE

(71) Applicant: ADVANCED SUTURE, INC., Dorado, PR (US)

(72) Inventors: Gregory A. Dumanian, Chicago, IL (US); Justin Herbert, Plymouth, MN (US); Adom Dumanian, San Juan, PR (US)

(21) Appl. No.: 16/114,482

(22) Filed: Aug. 28, 2018

Related U.S. Application Data
(63) Continuation of application No. 15/556,831, filed on Sep. 8, 2017.
(60) Provisional application No. 62/134,099, filed on Mar. 17, 2015.

(51) Int. Cl. A61B 17/06; A61B 17/04; A61B 17/00

(52) U.S. Cl. CPC A61B 17/06166; A61B 17/0487; A61B 17/06066; A61B 17/0401; A61B 2017/0608; A61B 2017/06052; A61B 2017/00004; A61B 2017/06171; A61B 2017/06176; A61B 2017/06185; A61B 2017/0464

(57) ABSTRACT

A medical device includes a surgical needle and an elongated suture. The suture has a first end attached to the surgical needle and a second end located away from the surgical needle. The suture includes a mesh wall and a plurality of pores extending through the mesh wall. At least some of the pores in the macroporous size range of greater than 200 microns and adapted to facilitate tissue integration through the mesh wall of the suture when introduced into a body. The suture is configured to resist a tensile load applied along an axial direction between the first and second ends of the elongated suture such that upon overloading the mesh wall collapses upon itself resulting in apparent suture elongation, collapsed diameter, and/or reduction in pore size.

### CROSS-REFERENCE TO RELATED APPLICATIONS

This is a continuation of U.S. application Ser. No. 15/556,831, filed Sep. 8, 2017, which is the US national phase of International Patent Application No. PCT/US16/20231, filed Mar. 1, 2016, which claims priority to U.S. Provisional Application No. 62/134,099, filed Mar. 17, 2015, the entire contents of each of which are expressly incorporated herein by reference.

### FIELD OF THE DISCLOSURE

The present disclosure is directed to sutures having structural characteristics that strengthen closure, prevent suture pull-through, and/or resist infection, and methods of use thereof.

### BACKGROUND

One of the foundations of surgery is the use of sutures to re-appose soft tissue, i.e., to hold tissue in a desired configuration until it can heal. In principle, suturing constitutes introducing a high tensile foreign construct (looped suture) into separate pieces of tissue in order to hold those pieces in close proximity until scar formation can occur, establishing continuity and strength between tissues. Sutures initially provide the full strength of the repair, but then become secondarily reinforcing or redundant as the tissue heals. The time until tissue healing reaches its maximal strength and is dependent on suture for approximation, therefore, is a period of marked susceptibility to failure of the repair due to forces naturally acting to pull the tissues apart.

Conventional sutures provide a circular or single-point cross-sectional profile extended over the length of the suture material. Such a suture has the great benefit of radial symmetry, which eliminates directional orientation, allowing the user (e.g., physician, surgeon, medic, etc.) to not have to worry about orienting the suture during use. However, a considerable disadvantage of the currently used single-point cross-section is that it does not effectively distribute force, and actively concentrates force at a geometric point (e.g., the point at the leading edge of the circle) creating a sharp edge in the axial dimension. Under these conditions, the tissue is continuously exposed to tension, increasing the likelihood that stress concentration at a geometric point or sharp edge will cut through the tissue.

Indeed, studies of surgical closures, a most prominent example being hernia repairs, demonstrate that the majority of failures or dehiscences occur in the early post-operative period, in the days, weeks, or months immediately following the operation, before full healing can occur. Sutures used to close the abdominal wall have high failure rates as demonstrated by the outcome of hernia formation. After a standard first-time laparotomy, the postoperative hernia occurrence rate is between 11-23%. The failure rate of sutures after hernia repair is as high as 54%. This is a sizeable and costly clinical problem, with approximately 200,000 post-operative incisional hernia repairs performed annually in the United States. Surgical failures have been blamed on poor suture placement, suture composition, patient issues such as smoking and obesity, and defects in cellular and extracellular matrices. Clinical experience in examining the cause of these surgical failures reveals that it is not breakage of suture as is commonly thought; in the majority of cases the cause is tearing of the tissue around the suture, or from another perspective, intact stronger suture cutting through weaker tissue. Mechanical analysis of the suture construct holding tissue together shows that a fundamental problem with current suture design is stress concentration at the suture puncture points through the tissue. That is, as forces act to pull tissues apart, rather than stress being more evenly distributed throughout the repair, it is instead concentrated at each point where the suture pierces through the tissue. The results are twofold: (1) constant stress at suture puncture points causes sliding of tissue around suture and enlargement of the holes, leading to loosening of the repair and an impairment of wound healing, and (2) at every puncture point where the stress concentration exceeds the mechanical strength of the tissue, the suture slices through the tissue causing surgical dehiscence. In addition, high pressure on the tissue created during tightening of the surgical knot can lead to local tissue dysfunction, irritation, inflammation, infection, and in the worst case tissue necrosis. This tissue necrosis found within the suture loop is one additional factor of eventual surgical failure.

There has been no commercial solution to the aforementioned problems with conventional sutures. Rather, thinner sutures continue to be preferred because it is commonly thought that a smaller diameter may minimize tissue injury. However, the small cross-sectional diameter in fact increases the local forces applied to the tissue, thereby increasing suture pull-through and eventual surgical failure.

For thousands of years conventional sutures have generally constituted thin solid lines of material, which unfortunately tear through the adjacent tissue when subject to large tensile loads present, for example, in hernia repair. There has been a persistent and long felt but unsolved need in the art of surgery for a suture that is capable of withstanding high tensile loads without tearing through the adjacent tissue—a problem known as “suture pull through”—in all types of surgical repair.

We are unaware of any suture in the art that solves the problem of “suture pull through” in all types of surgical repair. We are aware of the following products, which have tangentially attempted to address the problem of suture pull through and to improve the hold of tissue by sutures: barbed sutures, elastic sutures, zip ties, and felt pledgets. But none of these designs have become commonplace and accepted across all surgical disciplines. Barbed sutures exhibit improved tissue hold, but remain thin lines subject to conventional suture pull through. Under tension, elastic sutures stretch in an attempt to avoid pull through, but they also reduce in thickness, which is akin to sharpening a knife. Zip ties and felt pledgets have increased thicknesses for distributing forces and avoiding pull through, but are not sutures at all and, moreover, cannot be handled like sutures. The disclosed porous suture solves the long felt need (i.e., is capable of withstanding high tensile loads without tearing through the adjacent tissue) by providing a macroporous suture that uses “tissue incorporation” to promote healing in, around, and through the suture, thereby resulting in the scar tissue and the suture working together to form a stronger repair site than otherwise possible with conventional sutures. The disclosed porous suture has shown dramatic improvements in tissue holding ability as well as tissue incorporation in the laboratory and in experimental high-tension animal closures. See, e.g., (a) Dumanian et al., EXPERIMENTAL STUDY OF THE CHARACTERISTICS OF A NOVEL MESH SUTURE, British Journal of Surgery, Wiley Online Library, DOI: 10.1002/bjs.9853, Apr. 8, 2015, and (b) Petter-Puchner AH, THE STATE OF MIDLINE CLOSURE OF THE ABDOMINAL WALL British Journal of Surgery 102: 1446-1447, 2015.

The porous suture disclosed herein resists twice the magnitude of load before pulling through the adjacent tissue as that of conventional sutures. This exhibits a vast improvement in tissue holding ability that can predictably improve the administration of health care services across all surgical disciplines that require sutures and reduce incidents of follow-up surgeries and the burdensome costs associated therewith. Those of ordinary skill in the art of surgery have a natural bias against using thicker sutures that might distribute stresses because they increase the body's natural inflammatory response, which can lead to suture rejection, and they are more difficult to manipulate and produce palpable knots. The porous suture disclosed herein, however, unexpectedly results in a suture that takes advantage of the body's natural healing response by encouraging tissue growth in, around, and through the entire suture. Tissue incorporation of implanted foreign materials is well known to improve biocompatibility and to reduce the chance of delayed infections.

The porous suture of the present disclosure further unexpectedly results in a suture that is easily manipulated through tissue due to the tubular mesh construct, which allows the suture to deform and collapse under compressive forces. The porous suture disclosed herein still further unexpectedly results in a suture with improved knot characteristics due to its multi-filament tubular mesh construct. With tying, the area between filaments collapses for a low profile knot that holds well. Those skilled in the art know that multi-filament sutures have improved knot-holding characteristics in comparison to monofilament sutures.

One alternative to the conventional suture is disclosed by Calvin H. Frazier in U.S. Pat. No. 4,034,763. The Frazier patent discloses a tubular suture manufactured from loosely woven or expanded plastic material that has sufficient microporosity to be penetrated with newly formed tissue after introduction into the body. The Frazier patent does not expressly describe what pore sizes fall within the definition of “microporosity” and moreover it is not very clear as to what tissue “penetration” means. The Frazier patent does, however, state that the suture promotes the formation of ligamentous tissue for initially supplementing and then ultimately replacing the suture's structure and function. Furthermore, the Frazier patent describes that the suture is formed from Dacron or polytetrafluoroethylene (i.e., Teflon®), which are both commonly used as vascular grafts. From this disclosure, a person having ordinary skill in the art would understand that the suture disclosed in the Frazier patent would have pore sizes similar to those found in vascular grafts constructed from Dacron or Teflon®. It is well understood that vascular grafts constructed of these materials serve to provide a generally fluid-tight conduit for accommodating blood flow. Moreover, it is well understood that such materials have a microporosity that enables textured fibrous scar tissue formation adjacent to the graft wall such that the graft itself becomes encapsulated in that scar tissue. Tissue does not grow through the graft wall, but rather, grows about the graft wall in a textured manner. Enabling tissue in-growth through the wall of a vascular graft would be counterintuitive because vascular grafts are designed to carry blood; thus, porosity large enough to actually permit either leakage of blood or in-growth of tissue, which would restrict or block blood flow, would be counterintuitive and not contemplated. As such, these vascular grafts, and therefore the small pore sizes of the microporous suture disclosed in the Frazier patent, operate to discourage and prevent normal neovascularization and tissue in-growth into the suture. Pore sizes less than approximately 200 microns are known to be watertight and disfavor neovascularization. See, e.g., Mühl et al., New Objective Measurement to Characterize the Porosity of Textile Implants, Journal of Biomedical Materials Research Part B: Applied Biomaterials DOI 10.1002/jbmb, Page 5 (Wiley Periodicals, Inc. 2007). Accordingly, one skilled in the art would understand that the suture disclosed in the Frazier patent has a pore size that is at least less than approximately 200 microns. Thus, in summary, the Frazier patent seeks to take advantage of that microporosity to encourage the body's natural “foreign body response” of inflammation and scar tissue formation to create a fibrous scar about the suture.

Another alternative construct is disclosed by Wong in U.S. Patent Publication No. 2011/0137419, entitled “BIOCOMPATIBLE TANTALUM FIBER SCAFFOLDING FOR BONE AND SOFT TISSUE PROSTHESIS.” Wong discusses a suture constructed from a slurry of small metal filaments. Wong teaches (1) a method of making very small metal filaments, (2) a porous mat constructed of such filaments, and (3) a suture constructed of such filaments. The mat disclosed by Wong has pores between 100 microns and 500 microns. See Wong at para. [0021]. The suture disclosed by Wong is constructed by twisting the fibers together. See Wong at para. [0022]. Thus, to the extent that Wong teaches a suture, a person of ordinary skill in the art of surgery understands that such a suture would be constructed by twisting fibers together to form a solid, non-tubular, and non-porous construct. See Wong at para. [0022]. A person of ordinary skill in the art of surgery understands that by teaching a solid, non-porous suture in the same document that teaches a porous mat, Wong lacks and otherwise destroys any suggestion toward making a suture with porosity similar to the disclosed mat. It would not have been obvious to one of ordinary skill in the art of surgery to modify Frazier to include pores in the range of 100 microns to 500 microns, as disclosed in connection with the mat of Wong, because Wong's express teaching of a microsolid, non-tubular, and non-porous suture evidences that there would have been no expectation of success.

### GENERAL DESCRIPTION

In contrast to conventional sutures and to that disclosed by Frazier and Wong, the present disclosure is directed to sutures designed to discourage that “foreign body response” of inflammation and fibrotic tissue formation about the suture by utilizing a substantially macroporous structure over 200 microns that is also advantageously equipped with anti-roping elements. The macroporous structure seeks to minimize the foreign body response to the suture, while the anti-roping elements facilitate maintenance of the desired structural configuration of the suture when exposed to axial tensile loads, e.g., while the suture is being threaded into soft tissue. These anti-roping elements, however, do not prevent the suture from flattening with lateral loading. “Roping” is a phenomenon in the weaving industry whereby woven, knitted, or braided mesh materials tend to elongate under tension. This elongation can cause the various elements that make up the mesh material to collapse relative to each other and thereby reduce (e.g., close) the size of the pores disposed in the mesh. As such, the “anti-roping” elements of the present disclosure advantageously resist this elongation of the mesh suture and collapsing of the pores when the suture experiences axial tensile loads. By maintaining the desired structural configuration of the mesh suture during and after threading into soft tissue, the outer wall pores remain appropriately sized to facilitate tissue integration and/or prevent suture pull through.

### BRIEF DESCRIPTION OF THE DRAWINGS

FIG. 1 is a perspective view of an alternative suture constructed in accordance with the present application, and including anti-roping elements.

FIGS. 2 and 3 are detailed views of the mesh wall of the suture of FIG. 1.

FIG. 4 is a cross-sectional view of the mesh wall of the suture of FIG. 1 taken through line 4-4 of FIG. 3.

### DETAILED DESCRIPTION

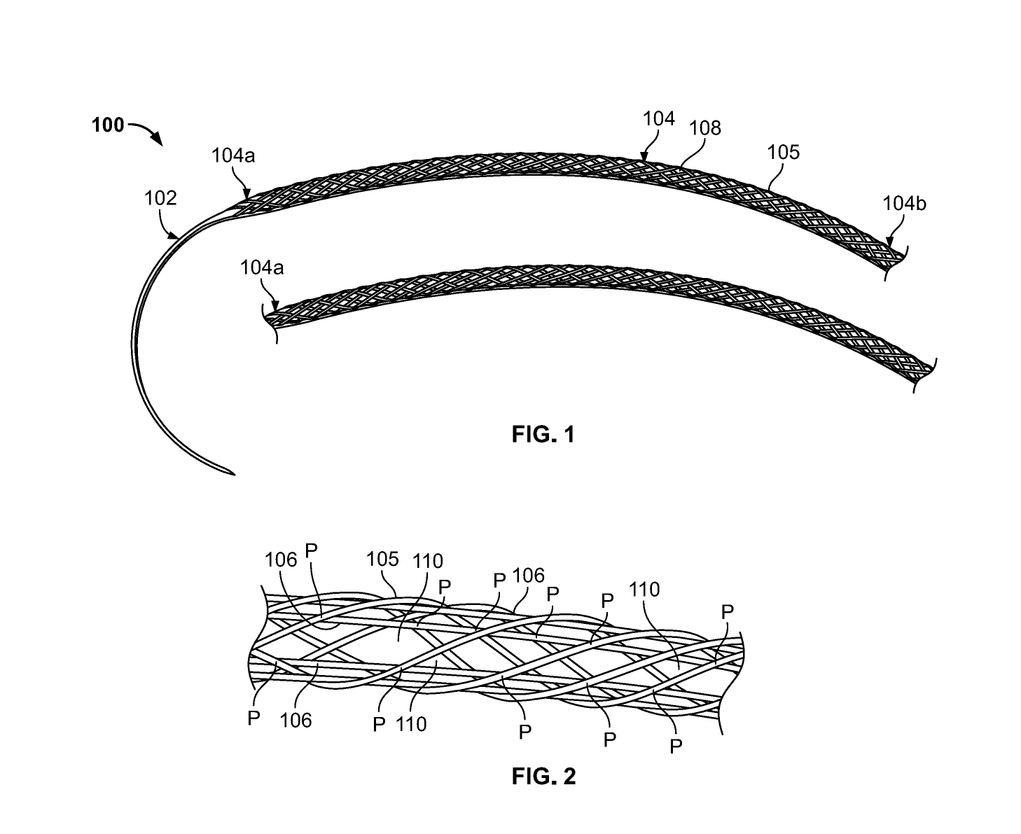

The present disclosure provides a medical suture having a macroporous construct that advantageously promotes neovascularization and normal tissue in-growth and integration subsequent to introduction into the body. In relation to FIGS. 1-4, the subject medical suture also includes anti-roping elements (e.g., longitudinally fixed elements) affixed to the macroporous material for resisting elongation and collapsing of pore size under tensile loads.

Additionally, the present disclosure provides various sutures with increased surface area, tissue integrative properties, cellular healing properties, and methods of use and manufacture thereof. In particular, provided herein are sutures with cross-section profiles and other structural characteristics that strengthen closure, prevent suture pull-through, and/or resist infection, and methods of use thereof. In some embodiments, sutures are provided that strengthen closure, prevent suture pull-through, and/or resist infection by, for example: (1) having a cross sectional profile that reduces pressure at suture points, (2) having a structural composition that allows tissue in-growth into the suture, or both (1) and (2). The present disclosure is not limited by any specific means for achieving the desired ends.

In some embodiments, conventional sutures exhibit a cross-sectional profile with radial symmetry or substantially radial symmetry. As used herein, the term “substantially radial symmetry” refers to a shape (e.g., cross-sectional profile) that approximates radial symmetry. A shape that has dimensions that are within 10% error of a shape exhibiting precise radial symmetry is substantially radially symmetric. For example, an oval that is 1.1 mm high and 1.0 mm wide is substantially radially symmetric. In some embodiments, the present disclosure provides sutures that lack radial symmetry and/or substantial radial symmetry.

In some embodiments, sutures are provided comprising cross-section shapes (e.g. flat, elliptical, etc.) that reduce tension against the tissue at the puncture site and reduce the likelihood of tissue tear. In some embodiments, devices (e.g., sutures) and methods provided herein reduce suture stress concentration at suture puncture points. In some embodiments, sutures with shaped cross-sectional profiles distribute forces more evenly (e.g., to the inner surface of the suture puncture hole) than traditional suture shapes/confirmation. In some embodiments, cross-sectionally-shaped sutures distribute tension about the suture puncture points. In some embodiments, rather than presenting a sharp point or line of suture to tissue, as is the case with traditional sutures, the sutures described herein present a flat or gently rounded plane to the leading edge of tissue, thereby increasing the surface area over which force can be distributed. In some embodiments, one cross-sectional dimension of the suture is greater than the orthogonal cross-sectional dimension (e.g., 1.1× greater, 1.2× greater, 1.3× greater, 1.4× greater, 1.5× greater, 1.6× greater, 1.7× greater, 1.8× greater, 1.9× greater, >2× greater, 2.0× greater, 2.1× greater, 2.2× greater, 2.3× greater, 2.4× greater, 2.5× greater, 2.6× greater, 2.7× greater, 2.8× greater, 2.9× greater, 3.0× greater, >3.0× greater, 3.1× greater, 3.2× greater, 3.3× greater, 3.4× greater, 3.5× greater, 3.6× greater, 3.7× greater, 3.8× greater, 3.9× greater, 4.0× greater, >4.0× greater . . . >5.0× greater . . . >6.0× greater . . . >7.0× greater . . . >8.0× greater . . . >9.0× greater . . . >10.0× greater). In some embodiments, sutures provided herein are flat or ellipsoidal on cross section, forming a ribbon-like conformation. In some embodiments, sutures are provided that do not present a sharp leading edge to the tissue. In some embodiments, use of the sutures described herein reduces the rates of surgical dehiscence in all tissues (e.g., hernia repairs, etc.). In some embodiments, sutures are provided with cross-sectional profiles that provide optimal levels of strength, flexibility, compliance, macroporosity, and/or durability while decreasing the likelihood of suture pull-through. In some embodiments, sutures are provided with sizes or shapes to enlarge the suture/tissue interface of each suture/tissue contact point, thereby distributing force over a greater area.

In some embodiments, sutures of the present disclosure provide various improvements over conventional sutures. In some embodiments, sutures provide: reduced likelihood of suture pull-through, increased closure strength, decreased number of stitches for a closure, more rapid healing times, and/or reduction in closure failure relative to a traditional suture. In some embodiments, relative improvements in suture performance (e.g., initial closure strength, rate of achieving tissue strength, final closure strength, rate of infection, etc.) are assessed in a tissue test model, animal test model, simulated test model, in silico testing, etc. In some embodiments, sutures of the present disclosure provide increased initial closure strength (e.g., at least a 10% increase in initial closure strength (e.g., >10%, >25%, >50%, >75%, >2-fold, >3-fold, >4-fold, >5-fold, >10-fold, or more). As used herein, “initial closure strength” refers to the strength of the closure (e.g., resistance to opening), prior to strengthening of the closure by the healing or scarring processes. In some embodiments, the increased initial closure strength is due to mechanical distribution of forces across a larger load-bearing surface area that reduces micromotion and susceptibility to pull through. In some embodiments, sutures of the present disclosure provide increased rate of achieving tissue strength (e.g., from healing of tissue across the opening, from ingrowth of tissue into the integrative (porous) design of the suture, etc.). In some embodiments, sutures of the present disclosure provide at least a 10% increase in rate of achieving tissue strength (e.g., >10%, >25%, >50%, >75%, >2-fold, >3-fold, >4-fold, >5-fold, >10-fold, or more). In some embodiments, increased rate of return of tissue strength across the opening further increases load bearing surface area, thereby promoting tissue stability and decreased susceptibility to pull through. In some embodiments, sutures of the present disclosure establish closure strength earlier in the healing process (e.g., due to greater initial closure strength and/or greater rate of achieving tissue strength) when the closure is most susceptible to rupture (e.g., at least a 10% reduction in time to establish closure strength (e.g., >10% reduction, >25% reduction, >50% reduction, >75% reduction, >2-fold reduction, >3-fold reduction, >4-fold reduction, >5-fold reduction, >10-fold reduction, or more)). In some embodiments, sutures of the present disclosure provide increased final closure strength (e.g., at least a 10% increase in final closure strength (e.g., >10%, >25%, >50%, >75%, >2-fold, >3-fold, >4-fold, >5-fold, >10-fold, or more). In some embodiments, the strength of fully healed closure is created not only by interface between the two apposed tissue surfaces, as is the case with conventional suture closures, but also along the total surface area of the integrated suture. In some embodiments, tissue integration into the suture decreases the rate of suture abscesses and/or infections that otherwise occur with solid foreign materials of the same size (e.g., at least a 10% reduction in suture abscesses and/or infection (e.g., >10% reduction, >25% reduction, >50% reduction, >75% reduction, >2-fold reduction, >3-fold reduction, >4-fold reduction, >5-fold reduction, >10-fold reduction, or more)). In some embodiments, sutures provide at least a 10% reduction (e.g., >10%, >20%, >30%, >40%, >50%, >60%, >70%, >80%, >90%, or more) in suture pull-through (e.g. through tissue (e.g., epidermal tissue, peritoneum, adipose tissue, cardiac tissue, or any other tissue in need of suturing), or through control substance (e.g., ballistic gel)).

In some embodiments, sutures are provided with any suitable cross-section profile or shape that provides reduced stress at the tissue puncture site, point of contact with tissue, and/or closure site. In some embodiments, sutures have cross-sectional dimensions (e.g., width and/or depth) or between 0.1 mm and 1 cm (e.g., 0.1 mm . . . 0.2 mm . . . 0.5 mm . . . 1.0 mm . . . 2.0 mm . . . 5.0 mm . . . 1 cm). In some embodiments, the suture dimensions (e.g., width and/or depth) that minimize pull-through and/or provide maximum load are utilized. In some embodiments, optimal suture dimensions are empirically determined for a given tissue and suture material. In some embodiments, one or both cross-sectional dimensions of a suture are the same as the cross-sectional dimensions of a traditional suture. In some embodiments, a suture comprises the same cross-sectional area as a traditional suture, but with different shape and/or dimensions. In some embodiments, a suture comprises the greater cross-sectional area than a traditional suture. In some embodiments, a suture cross-section provides a broad leading edge to spread pressure out over a broader portion of tissue. In some embodiments, a suture cross-section provides a shaped leading edge (e.g., convex) that evenly distributes force along a segment of tissue, rather than focusing it at a single point. In some embodiments, shaped sutures prevent pull-through by distributing forces across the tissue rather than focusing them at a single point. In some embodiments, sutures prevent pull-through by providing a broader cross-section that is more difficult to pull through tissue.

In some embodiments, ribbon-like suture or flat sutures are provided. In some embodiments, sutures provided herein comprise any suitable cross-sectional shape that provides the desired qualities and characteristics. In some embodiments, suture cross-sectional shape provides enhanced and/or enlarged leading edge surface distance and/or area (e.g. to reduce localized pressure on tissue). In some embodiments, suture cross-sectional shape comprises: an ellipse, half-ellipse, half-circle, gibbous, rectangle, square, crescent, pentagon, hexagon, concave ribbon, convex ribbon, H-beam, I-beam, dumbbell, etc. In some embodiments, a suture cross-sectional profile comprises any combination of curves, lines, corners, bends, etc. to achieve a desired shape. In some embodiments, the edge of the sutures configured to contact the tissue and/or place pressure against the tissue is broader than one or more other suture dimensions. In some embodiments, the edge of the sutures configured to contact the tissue and/or place pressure against the tissue is shaped to evenly distribute forces across the region of contact.

In some embodiments, hollow core sutures are provided such as that depicted in FIG. 1. More specifically, FIG. 1 depicts a medical device 100 that includes a surgical needle 102 and an elongated suture 104. In FIG. 1, the needle 102 includes a contoured or curved needle with a flattened cross-sectional profile, but needles with generally any geometry could be used. The suture 104 can be a hollow core suture with a first end 104a attached to the needle 102 and a second end 104b located a distance away from the needle 102. In some embodiments, the needle 102 can be directly attached to the suture 104. In some other embodiments, the needle 102 can be indirectly attached to the suture 104 by way of an intervening component such as a permanent connecting mechanism or a removable connecting mechanism. An example of a permanent connection mechanism might include a physical bridge (e.g., a rod, a bar, a pin, a collar, etc.) or other such intervening component disposed between the needle 102 and the suture 104, wherein one portion (e.g., a first end) of the component is permanently affixed to the suture 104 and another portion (e.g., a second end) of the component is permanently affixed to the needle 102. An example of a removable connecting mechanism may be any connecting mechanism that a user can easily affix or remove the needle 102 from the suture 104 or vice versa. For example, in some embodiments, a removable connecting mechanism might include a hook or ball or barb structure with one end permanently affixed to an end of the suture 104, and a second end formed in the shape of a hook or ball or barb for being received in an eyelet of the needle 102. These are only examples of intervening components that might be implements in order to achieve attachment between the needle 102 and the suture 104 of the present disclosure. Other possibilities exist and are intended to be within the scope of the present disclosure.

As shown in FIG. 1, the entire length of the suture 104 between the first and second ends 104a, 104b can include a tubular wall 105 that defines a hollow core 108. In other versions, however, less than the entire length of the suture 104 can be tubular. For example, it is foreseeable that either or both of the first and second ends 104a, 104b can have a non-tubular portion or portion of other geometry. Such non-tubular portions could be for attaching the first end 104a of the suture 104 to the needle 102 or for tying off the second end 104b, for example. In versions where the entire length of the suture 14 is tubular, as shown, the entire length of the suture 104 including the ends and central portion also has generally a constant or uniform diameter or thickness in the absence of stresses. That is, no portion of the suture 104 is meaningfully larger in diameter than any other portion of the suture 104. Moreover, no aspect, end, or other portion of the suture 104 is intended to be or is actually passed through, disposed in, received in, or otherwise positioned inside of the hollow core 108. The hollow core 108 is adapted for receiving tissue in-growth only.

In some embodiments, the tubular wall 105 can have a length that is greater than or equal to approximately 20 cm, greater than or equal to approximately 30 cm, greater than or equal to approximately 40 cm, greater than or equal to approximately 50 cm, greater than or equal to approximately 60 cm, greater than or equal to approximately 70 cm, greater than or equal to approximately 80 cm, greater than or equal to approximately 90 cm, and/or greater than or equal to approximately 100 cm, or even bigger. In some embodiments, the tubular wall 106 can have a diameter in a range of approximately 1 mm to approximately 10 mm and can be constructed of a material such as, for example, polyethylene terephthalate, nylon, polyolefin, polypropylene, silk, polymers p-dioxanone, co-polymer of p-dioxanone, ε-caprolactone, glycolide, LH-lactide, D(+)-lactide, meso-lactide, trimethylene carbonate, polydioxanone homopolymer, and combinations thereof. So constructed, the tubular wall 105 of the suture 104 can be radially deformable such that it adopts a first cross-sectional profile in the absence of lateral stresses and a second cross-sectional profile in the presence of lateral stresses. For example, in the absence of lateral stresses, the tubular wall 105 and therefore the suture 104 depicted in FIG. 1, for example, can have a circular cross-sectional profile, thereby exhibiting radial symmetry. In the presence of a lateral stress, such a suture 104 could then exhibit a partially or wholly collapsed conformation. The stiffness of the materials may vary from a suture that completely collapses with lateral stress, to a suture that retains a its original profile with lateral stress.

In at least one version of the medical device 100, at least some of the tubular wall 106 can be macroporous defining a plurality of pores 110 (e.g., openings, apertures, holes, etc.), only a few of which are expressly identified by reference number and lead line in FIG. 10 for clarity. The pores 110 extend completely through the mesh wall 105 to the hollow core 108. In some versions, the tubular wall 105 can be constructed of a woven or knitted mesh material. In one version, the wall 105 can be constructed of a knitted mesh material used in abdominal wall hernia repair.

As used herein, the term “macroporous” can include pore sizes that are at least greater than or equal to approximately 200 microns and, preferably, greater than or equal to 500 microns. In some versions of the medical device 100, the size of at least some the pores 110 in the suture 104 can be in a range of approximately 500 microns to approximately 4 millimeters. In another version, at least some of the pores 110 can have a pore size in the range of approximately 500 microns to approximately 2.5 millimeters. In another version, at least some of the pores 110 can have a pore size in the range of approximately 1 millimeter to approximately 2.5 millimeters. In another version, the size of at least some of the pores 110 can be approximately 2 millimeters. Moreover, in some versions, the pores 110 can vary in size. Some of the pores 110 can be macroporous (e.g., greater than approximately 200 microns) and some of the pores 110 can be microporous (e.g., less than approximately 200 microns). The presence of microporosity (i.e., pores less than approximately 200 microns) in such versions of the disclosed suture may only be incidental to the manufacturing process, which can including knitting, weaving, extruding, blow molding, or otherwise, but not necessarily intended for any other functional reason regarding biocompatibility or tissue integration. The presence of microporosity (i.e, some pores less than approximately 200 microns in size) as a byproduct or incidental result of manufacturing does not change the character of the disclosed macroporous suture (e.g., with pores greater than approximately 200 microns, and preferably greater than approximately 500 microns, for example), which facilitates tissue in-growth to aid biocompatibility, reduce tissue inflammation, and decrease suture pull-through.

In versions of the disclosed suture that has both macroporosity and microporosity, the number of pores 110 that are macroporous can be in a range from approximately 1% of the pores to approximately 99% of the pores (when measured by pore cross-sectional area), in a range from approximately 5% of the pores to approximately 99% of the pores (when measured by pore cross-sectional area), in a range from approximately 10% of the pores to approximately 99% of the pores (when measured by pore cross-sectional area), in a range from approximately 20% of the pores to approximately 99% of the pores (when measured by pore cross-sectional area), in a range from approximately 30% of the pores to approximately 99% of the pores (when measured by pore cross-sectional area), in a range from approximately 50% of the pores to approximately 99% of the pores (when measured by pore cross-sectional area), in a range from approximately 60% of the pores to approximately 99% of the pores (when measured by pore cross-sectional area), in a range from approximately 70% of the pores to approximately 99% of the pores (when measured by pore cross-sectional area), in a range from approximately 80% of the pores to approximately 99% of the pores (when measured by pore cross-sectional area), or in a range from approximately 90% of the pores to approximately 99% of the pores (when measured by pore cross-sectional area).

So configured, the pores 110 in the suture 104 are arranged and configured such that the suture 104 is adapted to facilitate and allow tissue in-growth and integration through the pores 110 in the mesh wall 105 and into the hollow core 108 when introduced into a body. That is, the pores 110 are of sufficient size to achieve maximum biocompatibility by promoting local/normal tissue in-growth through the pores 110 and into the hollow core 108 of the suture 104. As such, tissue growth through the pores 110 and into the hollow core 108 enables the suture 104 and resultant tissue to combine and cooperatively increase the strength and efficacy of the medical device 100, while also decreasing irritation, inflammation, local tissue necrosis, and likelihood of pull through. Instead, the suture 14 promotes the production of healthy new tissue throughout the suture construct including inside the pores 110 and the hollow core 108.

While the suture 104 in FIG. 1 has been described as including a single elongated hollow core 108, in some embodiments, a suture according to the present disclosure can comprise a tubular wall defining a hollow core including one or more interior voids (e.g., extending the length of the suture). In some versions, at least some of the interior voids can have a size or diameter>approximately 200 microns, >approximately 300 microns, >approximately 400 microns, >approximately 500 microns, >approximately 600 microns, >approximately 700 microns, >approximately 800 microns, >approximately 900 microns, >approximately 1 millimeter, or >approximately 2 millimeters. In some embodiments, a suture according to the present disclosure can comprise a tubular wall defining a hollow core including one or more (e.g., 1, 2, 3, 4, 5, 6, 7, 8, or more) lumens (e.g., running the length of the suture). In some embodiments, a suture according to the present disclosure can comprise a tubular wall defining a hollow core including a honeycomb structure, a 3D lattice structure, or other suitable interior matrix, which defines one or more interior voids. In some versions, at least some of the interior voids in the honeycomb structure, 3D lattice structure, or other suitable matrix can have a size or diameter>approximately 200 microns, >approximately 300 microns, >approximately 400 microns, >approximately 500 microns, >approximately 600 microns, >approximately 700 microns, >approximately 800 microns, >approximately 900 microns, >approximately 1 millimeter, or >approximately 2 millimeters. In some embodiments, a void comprises a hollow core. In some embodiments, a hollow core can include a hollow cylindrical space in the tubular wall, but as described, the term “hollow core” is not limited to defining a cylindrical space, but rather could include a labyrinth of interior voids defined by a honeycomb structure, a 3D lattice structure, or some other suitable matrix. In some embodiments, sutures comprise a hollow, flexible structure that has a circular cross-sectional profile in its non-stressed state, but which collapses into a more flattened cross-sectional shape when pulled in an off-axis direction. In some embodiments, sutures are provided that exhibit radial symmetry in a non-stressed state. In some embodiments, radial symmetry in a non-stressed state eliminates the need for directional orientation while suturing. In some embodiments, sutures are provided that exhibit a flattened cross-sectional profile when off-axis (longitudinal axis) force is applied (e.g., tightening of the suture against tissue), thereby more evenly distributing the force applied by the suture on the tissue. In some embodiments, sutures are provided that exhibit a flattened cross-sectional profile when axial force is applied. In some embodiments, sutures comprise flexible structure that adopts a first cross-sectional profile in its non-stressed state (e.g., suturing profile), but adopts a second cross-sectional shape when pulled in an off-axis direction (e.g., tightened profile). In some embodiments, a suture is hollow and/or comprises one or more internal voids (e.g., that run the length of the suture). In some embodiments, internal voids are configured to encourage the suture to adopt a preferred conformation (e.g., broadened leading edge to displace pressures across the contacted tissue) when in a stressed states (e.g., tightened profile). In some embodiments, internal voids are configured to allow a suture to adopt radial exterior symmetry (e.g., circular outer cross-sectional profile) when in a non-stressed state. In some embodiments, varying the size, shape, and/or placement of internal voids alters one or both of the first cross-sectional profile (e.g., non-stressed profile, suturing profile) and second cross-sectional profile (e.g., off-axis profile, stressed profile, tightened profile). In some embodiments, an internal element is absorbed over time, rendering the space confined by the outer mesh changing as to shape and size. In some elements, the space confined by the outer mesh is used to deliver cells or medicaments for delivery to the tissues.

Sutures, which are substantially linear in geometry, have two distinct ends, as described above with reference to FIG. 1, for example. In some embodiments, both ends are identical. In some embodiments, each end is different. In some embodiments, one or both ends are structurally unadorned. In some embodiments, one or more ends is attached to or at least configured for attachment to a needle via swaging, sonic welding, adhesive, tying, or some other means (as shown FIG. 1). In some embodiments, the second end 104b of the suture 104 is configured to include an anchor for anchoring the suture 104 against the tissue through which the suture 104 is inserted. In some embodiments, the second end 104b of the suture 104 is configured to anchor the suture at the beginning of the closure. In some embodiments, the second end 104b of the suture 104 includes an anchor that is a structure that prevents the suture 104 from being pulled completely through the tissue. In some embodiments, the anchor has a greater dimension than the rest of the suture 104 (at least 10% greater, at least 25% greater, at least 50% greater, at least 2-fold greater, at least 3-fold greater, at least 4-fold greater, at least 5-fold greater, at least 6-fold greater, at least 10-fold greater, etc.). In some embodiments, the anchor comprises a structure with any suitable shape for preventing the suture 104 from being pulled through the hole (e.g., ball, disc, plate, cylinder), thereby preventing the suture 14 from being pulled through the insertion hole. In some embodiments, the anchor of the suture 104 comprises a closed loop. In some embodiments, the closed loop is of any suitable structure including, but not limited to a crimpled loop, flattened loop, or a formed loop. In some embodiments, a loop can be integrated into the end of the suture 104. In some embodiments, a separate loop structure can be attached to the suture 104. In some embodiments, the needle 102 can be passed through the closed loop anchor to create a cinch for anchoring the suture 104 to that point. In some embodiments, the anchor can comprise one or more structures (e.g., barb, hook, etc.) to hold the end of the suture 104 in place. In some embodiments, one or more anchor 22 structures (e.g., barb, hook, etc.) are used in conjunction with a closed loop to ratchet down the cinch and hold its position. In some embodiments, a knotless anchoring system can be provided. In some embodiments, a needle can be attached to the second end 104b to create a double armed suture. In some embodiments, a single mesh suture or multiple mesh sutures are attached to a larger device such as a reconstruction mesh or implant to aid in deployment of the larger device.

In some embodiments, and as briefly mentioned relative to FIG. 1, the present disclosure provides suturing needles with cross-sectional profiles configured to prevent suture pull-through and methods of use thereof. In some embodiments, suturing needles are provided comprising cross-section shapes (e.g. flat, elliptical, transitioning over the length of the needle, etc.) that reduce tension against the tissue at the puncture site and reduce the likelihood of tissue tear. In some embodiments, one cross-sectional dimension of the needle is greater than the orthogonal cross-sectional dimension (e.g., 1.1× greater, 1.2× greater, 1.3× greater, 1.4× greater, 1.5× greater, 1.6× greater, 1.7× greater, 1.8× greater, 1.9× greater, >2× greater, 2.0× greater, 2.1× greater, 2.2× greater, 2.3× greater, 2.4× greater, 2.5× greater, 2.6× greater, 2.7× greater, 2.8× greater, 2.9× greater, 3.0× greater, >3.0× greater, 3.1× greater, 3.2× greater, 3.3× greater, 3.4× greater, 3.5× greater, 3.6× greater, 3.7× greater, 3.8× greater, 3.9× greater, 4.0× greater, >4.0× greater . . . >5.0× greater . . . >6.0× greater . . . >7.0× greater . . . >8.0× greater . . . >9.0× greater . . . >10.0× greater). In some embodiments, suturing needles are provided circular in shape at its point (e.g., distal end), but transition to a flattened profile (e.g., ribbon-like) to the rear (e.g. proximal end). In some embodiments, the face of the flattened area is orthogonal to the radius of curvature of the needle. In some embodiments, suturing needles create a slit (or flat puncture) in the tissue as it is passed through, rather than a circle or point puncture. In some embodiments, suturing needles are provided circular in shape at its point (e.g., distal end), but transition to a 2D cross-sectional profile (e.g., ellipse, crescent, half moon, gibbous, etc.) to the rear (e.g. proximal end). In some embodiments, suturing needles provided herein find use with the sutures described herein. In some embodiments, suturing needles find use with sutures of the same shape and/or size. In some embodiments, suturing needles and sutures are not of the same size and/or shape. In some embodiments, suturing needles provided herein find use with traditional sutures. Various types of suture needles are well known in the art. In some embodiments, suturing needles provided herein comprise any suitable characteristics of suturing needles known to the field, but modified with dimensions described herein. Any introduction device of the mesh suture through tissue is defined as a needle, and therefore we do not limit our embodiments to those defined here, but rather any sharp instrument that can penetrate tissue to pass the suture.

In some embodiments, the present disclosure also provides compositions, methods, and devices for anchoring the suture at the end of the closure (e.g., without tying the suture to itself). In some embodiments, one or more securing elements (e.g., staples) are positioned over the terminal end of the suture to secure the end of the closure. In some embodiments, one or more securing elements (e.g., staples) are secured to the last “rung” of the suture closure (e.g., to hold the suture tight across the closure. In some embodiments, a securing element is a staple. In some embodiments, a staple comprises stainless steel or any other suitable material. In some embodiments, a staple comprises a plurality of pins that can pass full thickness through 2 layers of suture. In some embodiments, staple pins are configured to secure the suture end without cutting and/or weakening the suture filament. In some embodiments, a staple forms a strong joint with the suture. In some embodiments, a staple is delivered after the needle is cut from the suture. In some embodiments, a staple is delivered and the needle removed simultaneously

In some embodiments, the present disclosure provides devices (e.g., staple guns) for delivery of a staple into tissue to secure the suture end. In some embodiments, a staple deployment device simultaneously or near-simultaneously delivers a staple and removes the needle from the suture. In some embodiments, a staple deployment device comprises a bottom lip or shelf to pass under the last rung of suture (e.g., between the suture and tissue surface) against which the pins of the staple can be deformed into their locked position. In some embodiments, the bottom lip of the staple deployment device is placed under the last rung of suture, the free tail of the suture is placed within the stapling mechanism, and the suture is pulled tight. In some embodiments, while holding tension, the staple deployment device is activated, thereby joining the two layers of suture together. In some embodiments, the device also cuts off the excess length of the free suture tail. In some embodiments, the staple deployment device completes the running suture and trims the excess suture in one step. In some embodiments, a suture is secured without the need for knot tying. In some embodiments, only 1 staple is needed per closure. In some embodiments, a standard stapler is used to apply staples and secure the suture end. In some embodiments, a staple is applied to the suture end manually. The staple may or may not have tissue integrative properties.

In some embodiments, sutures provided herein provide tissue integrative properties to increase the overall strength of the repair (e.g., at an earlier time-point than traditional sutures). In some embodiments, sutures are provided with enhanced tissue adhesion properties. In some embodiments sutures are provided that integrate with the surrounding tissue. In some embodiments, tissue integrative properties find use in conjunction with any other suture characteristics described herein. In some embodiments, sutures allow integration of healing tissue into the suture. In some embodiments, tissue growth into the suture is promoted (e.g., by the surface texture of the suture). In some embodiments, tissue growth into the suture prevents sliding of tissue around suture, and/or minimizes micromotion between suture and tissue. In some embodiments, tissue in-growth into the suture increases the overall strength of the repair by multiplying the surface area for scar in establishing continuity between tissues. Conventionally, the strength of a repair is dependent only on the interface between the two tissue surfaces being approximated. In some embodiments in-growth of tissue into the suture adds to the surface area of the repair, thereby enhancing its strength. In some embodiments, increasing the surface area for scar formation, the closure reaches significant strength more quickly, narrowing the window of significant risk of dehiscence.

In some embodiments, the surface and/or internal texture of a suture promote tissue adhesion and/or ingrowth. In some embodiments, as discussed above specifically with reference to FIG. 1, a suture of the present disclosure can comprise a porous (e.g., macroporous) and/or textured material. In some embodiments, a suture comprises a porous (e.g., macroporous) and/or textured exterior. In some embodiments, pores in the suture allow tissue in-growth and/or integration. In some embodiments, a suture comprises a porous ribbon-like structure, instead of a tubular like structure. In some embodiments, a porous suture comprises a 2D cross-sectional profile (e.g., elliptical, circular (e.g., collapsible circle), half moon, crescent, concave ribbon, etc.). In some embodiments, a porous suture comprises polypropylene or any other suitable suture material. In some embodiments, pores are between 500 μm and 3.5 mm or greater in diameter (e.g., e.g., >500 μm in diameter (e.g., ,>500 μm, >600 μm , >700 μm , 800 μm, >900 μm, >1 mm, or more). In some embodiments pores are of varying sizes. In some embodiments, a suture comprises any surface texture suitable to promote tissue in-growth and/or adhesion. In some embodiments, suitable surface textures include, but are not limited to ribbing, webbing, mesh, barbs, grooves, etc. In some embodiments, the suture may include filaments or other structures (e.g., to provide increased surface area and/or increased stability of suture within tissue). In some embodiments, interconnected porous architecture is provided, in which pore size, porosity, pore shape and/or pore alignment facilitates tissue in-growth.

In some embodiments, a suture comprises a mesh and/or mesh-like exterior. In some embodiments, a mesh exterior provides a flexible suture that spreads pressure across the closure site, and allows for significant tissue in-growth. In some embodiments, the density of the mesh is tailored to obtain desired flexibility, elasticity, and in-growth characteristics.

In some embodiments, a suture is coated and/or embedded with materials to promote tissue ingrowth. Examples of biologically active compounds that may be used sutures to promote tissue ingrowth include, but are not limited to, cell attachment mediators, such as the peptide containing variations of the “RGD” integrin binding sequence known to affect cellular attachment, biologically active ligands, and substances that enhance or exclude particular varieties of cellular or tissue ingrowth. Such substances include, for example, osteoinductive substances, such as bone morphogenic proteins (BMP), epidermal growth factor (EGF), fibroblast growth factor (FGF), platelet-derived growth factor (PDGF), insulin-like growth factor (IGF-I and II), TGF-β, etc. Examples of pharmaceutically active compounds that may be used sutures to promote tissue ingrowth include, but are not limited to, acyclovir, cephradine, malfalen, procaine, ephedrine, adriomycin, daunomycin, plumbagin, atropine, guanine, digoxin, quinidine, biologically active peptides, chlorin e.sub.6, cephalothin, proline and proline analogues such as cis-hydroxy-L-proline, penicillin V, aspirin, ibuprofen, steroids, antimetabolites, immunomodulators, nicotinic acid, chemodeoxycholic acid, chlorambucil, and the like. Therapeutically effective dosages may be determined by either in vitro or in vivo methods.

Sutures are well known medical devices in the art. In some embodiments, sutures have braided or monofilament constructions. In some embodiments sutures are provided in single-armed or double-armed configurations with a surgical needle mounted to one or both ends of the suture, or may be provided without surgical needles mounted. In some embodiments, the end of the suture distal to the needle comprises one or more structures to anchor the suture. In some embodiments, the distal end of the suture comprises one or more of a: closed loop, open loop, anchor point, barb, hook, etc. In some embodiments, sutures comprise one or more biocompatible materials. In some embodiments, sutures comprise one or more of a variety of known bioabsorbable and nonabsorbable materials. For example, in some embodiments, sutures comprise one or more aromatic polyesters such as polyethylene terephthalate, nylons such as nylon 6 and nylon 66, polyolefins such as polypropylene, silk, and other nonabsorbable polymers. In some embodiments, sutures comprise one or more polymers and/or copolymers of p-dioxanone (also known as 1,4-dioxane-2-one), ε-caprolactone, glycolide, L(−)-lactide, D(+)-lactide, meso-lactide, trimethylene carbonate, and combinations thereof. In some embodiments, sutures comprise polydioxanone homopolymer. The above listing of suture materials should not be viewed as limiting. In some embodiments, the disclosed sutures can be constructed of metal filaments such as stainless steel filaments. Suture materials and characteristics are known in the art. Any suitable suture materials or combinations thereof are within the scope of the present disclosure. In some embodiments, sutures comprise sterile, medical grade, surgical grade, and or biodegradable materials. In some embodiments, a suture is coated with, contains, and/or elutes one or more bioactive substances (e.g., antiseptic, antibiotic, anesthetic, promoter of healing, etc.). In some embodiments, the suture filaments and or the hollow core 108 of any of the disclosed sutures can contain a drug product for delivery to the patient, the medicament could take the form of a solid, a gel, a liquid, or otherwise. In some embodiments, the suture filaments and or the hollow core 108 of any of the disclosed sutures can be seeded with cells or stem cells to promote healing, ingrowth or tissue apposition.

In some embodiments, the structure and material of the suture provides physiologically-tuned elasticity. In some embodiments, a suture of appropriate elasticity is selected for a tissue. In some embodiments, suture elasticity is matched to a tissue. For example, in some embodiments, sutures for use in abdominal wall closure will have similar elasticity to the abdominal wall, so as to reversibly deform along with the abdominal wall, rather than act as a relatively rigid structure that would carry higher risk of pull-through. In some embodiments, elasticity would not be so great however, so as to form a loose closure that could easily be pulled apart. In some embodiments, deformation of the suture would start occurring just before the elastic limit of its surrounding tissue, e.g., before the tissue starts tearing or irreversibly deforming.

In some embodiments, sutures described herein provide a suitable replacement or alternative for surgical repair meshes (e.g., those used in hernia repair). In some embodiments, the use of sutures in place of mesh reduces the amount of foreign material placed into a subject. In some embodiments, the decreased likelihood of suture pull-through allows the use of sutures to close tissues not possible with traditional sutures (e.g., areas of poor tissue quality (e.g., muscle tissue lacking fascia, friable or weak tissue) due to conditions like inflammation, fibrosis, atrophy, denervation, congenital disorders, attenuation due to age, or other acute and chronic diseases). Like a surgical mesh, sutures described herein permit a distribution of forces greater than that achieved by standard sutures delocalizing forces felt by the tissue and reducing the chance of suture pull-though and failure of the closure.

In some embodiments, sutures are permanent, removable, or absorbable. In some embodiments, permanent sutures provide added strength to a closure or other region of the body, without the expectation that the sutures will be removed upon the tissue obtaining sufficient strength. In such embodiments, materials are selected that pose little risk of long-term residency in a tissue or body. In some embodiments, removable sutures are stable (e.g., do not readily degrade in a physiological environment), and are intended for removal when the surrounding tissue reaches full closure strength. In some embodiments, absorbable sutures integrate with the tissue in the same manner as permanent or removable sutures, but eventually (e.g., >1 week, >2 weeks, >3 weeks, >4 weeks, >10 weeks, >25 weeks, >1 year) biodegrade and/or are absorbed into the tissue after having served the utility of holding the tissue together during the post-operative and/or healing period. In some embodiments absorbable sutures present a reduced foreign body risk.

Although prevention of dehiscence of abdominal closures (e.g., hernia formation) is specifically described at an application of embodiments of the present disclosure, the sutures described herein are useful for joining any tissue types throughout the body. In some embodiments, sutures described herein are of particular utility to closures that are subject to tension and/or for which cheesewiring is a concern. Exemplary tissues within which the present disclosure finds use include, but are not limited to: connective tissue, fascia, ligaments, muscle, dermal tissue, cartilage, tendon, or any other soft tissues. Specific applications of sutures described herein include reattachments, plication, suspensions, slings, etc. Sutures described herein find use in surgical procedures, non-surgical medical procedures, veterinary procedures, in-field medical procedures, etc. The scope of the present disclosure is not limited by the potential applications of the sutures described herein.

Yet, from the foregoing, it should also be appreciated that the present disclosure additionally provides both a novel method of re-apposing soft tissue and a novel method of manufacturing a medical device.

Based on the present disclosure, a method of re-apposing soft tissue can first include piercing a portion of the soft tissue with the surgical needle 102 attached to a first end 104a of a tubular suture 104. Next, a physician can thread the tubular suture 104 through the soft tissue and make one or more stitches, as is generally known. Finally, the physician can anchor the tubular suture 104 in place in the soft tissue. As disclosed hereinabove, the tubular suture 104 comprises a tubular mesh wall 105 defining a hollow core 108. The tubular mesh wall 106 defines a plurality or pores 110, each with a pore size that is greater than or equal to approximately 200 or 500 microns but with some smaller as to manufacturing. So configured, the tubular suture 104 is adapted to accommodate the soft tissue growing through the tubular mesh wall 106 and into the hollow core 108, thereby integrating with the suture. In some versions, the method can further and finally include anchoring the tubular suture 104 in place by passing the surgical needle 102 through a closed loop or anchor at the second end 104b of the tubular suture 104 and creating a cinch for anchoring the suture 104 to the soft tissue. Once anchored, the suture 104 can be cut off near the anchor and any remaining unused portion of the suture 104 can be discarded.

A method of manufacturing a medical device in accordance with the present disclosure can include forming a tubular wall 105 having a plurality or pores 110 and defining a hollow core 108, each pore 110 having a pore size that is greater than 200 microns. Additionally, the method of manufacturing can include attaching a first end 104a of the tubular wall 104 to a surgical needle 102. Forming the tubular wall 104 can include forming a tube from a mesh material. The tubular mesh wall 105 may be formed by directly weaving, braiding, or knitting fibers into a tube shape. Alternatively, forming the tubular mesh wall 16 can include weaving, braiding, or knitting fibers into a planar sheet and subsequently forming the planar sheet into a tube shape. Of course, other manufacturing possibilities including extrusion exist and twisting filaments are not the only possibilities for creating a porous tube within the scope of the present disclosure, but rather, are mere examples.

Still further, a method of manufacturing a medical device 100 in accordance with the present disclosure can include providing an anchor on an end of the tubular wall 105 opposite the needle 102. In some versions of the method, and as one example only, providing the anchor can be as simple as forming a loop.

In some embodiments, the tubular wall 105 can be divided into two or more tubular wall portions by one or more intervening features such as knots, inflexible rod-like members, monofilament or multi-filament suture segments, etc. Such a construct can be referred to as a segmented mesh suture constructed in accordance with the present disclosure

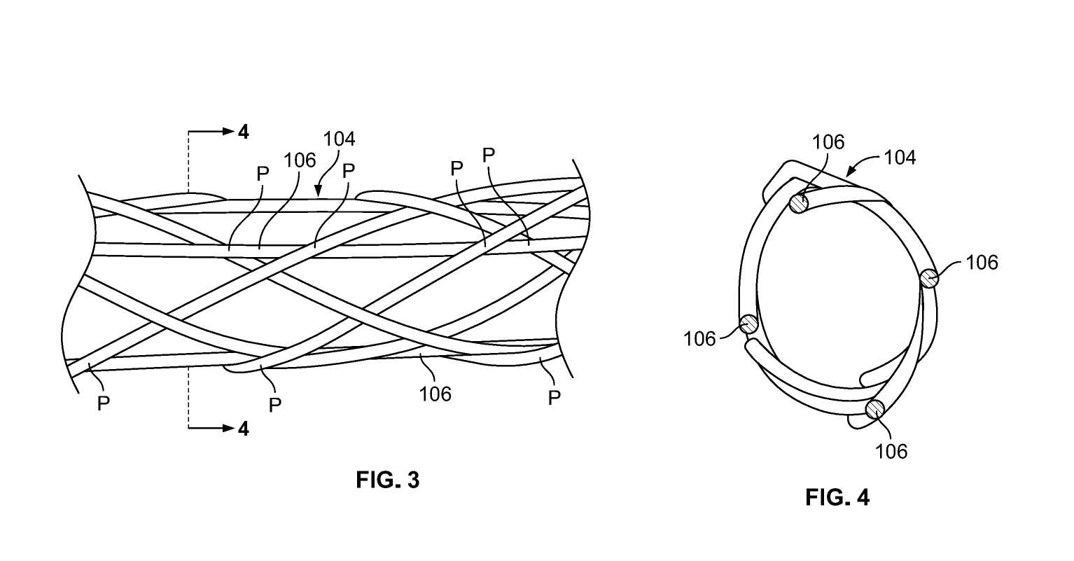

As mentioned, one optional feature of the medical device 100 of FIGS. 1-4 is that it can include one or more anti-roping elements 106. That is, the medical device 100 can include one or more, or a plurality of, anti-roping elements 106 in the form of elongated elements 106 extending substantially (or entirely) the entire length of the suture 104 between the first and second ends 104a, 104b. The elongated elements 106 are fixed (or are not fixed) to the mesh wall 105 of the suture 104 at a plurality of points P and thereby serve to resist elongation of the suture 104 upon the application of an axial tensile load to the medical device 100. In some embodiments, the elongated elements 106 can be fixed to the mesh wall 105 in any available manner including, without limitation, welding, gluing, tying, braiding, heating, staking, dipping, chemically bonding, etc. In some embodiments, the elongated elements 106 are not fixed to the helical filaments. In some embodiments, the various fibers/filaments that make up the mesh wall 105 of any of the sutures described herein can also be fixed together at the intersection between fibers/filaments in any available manner including, without limitation, welding, gluing, tying, braiding, heating, staking, dipping, chemically bonding, etc. As shown in FIG. 3, for example, the present version of the anti-roping elements 106 can be arranged such that each anti-roping element 106 is interleaved between adjacent elements of the remainder of the mesh suture 104, which can add to the integrity and stability of the suture 104. In other embodiments, the anti-roping elements 106 can be positioned entirely on an outer perimeter or on an inner perimeter of the tubular suture 104. In other embodiments, some of the elements 106 can be positioned on an inner perimeter, some can be positioned on an outer perimeter, and/or some can be interleaved such as depicted in FIG. 3. In other embodiments, some or all of the anti-roping elements may reside in the central core. In some embodiments, the anti-roping elements themselves are not entirely linear single filaments, but rather are a braid of fine filaments that act to run the length of the suture either obliquely or in step-wise fashion to resist elongation.

As mentioned above, “roping” is a phenomenon in the weaving industry whereby woven, braided, or knitted mesh materials tend to elongate under tension. This elongation can cause the various elements that make up the mesh material to collapse relative to each other and thereby reduce (e.g., close) the size of the pores disposed in the mesh. As such, the “anti-roping” elements 106 of the present disclosure, which are embodied as longitudinal elements in FIGS. 1-4, advantageously resist this elongation of the mesh suture and collapsing of the pores when the suture experiences axial tensile loads. This resistance is achieved because the anti-roping elements adds structural integrity to the overall construct and prevents the various mesh elements from moving relative to each other and/or deforming under tension. By maintaining the desired structural configuration of the mesh suture during and after threading into soft tissue, the pores remain appropriately sized to facilitate tissue integration and the overall width and/or dimension of the suture remains appropriately sized to limit and/or prevent suture pull through.

In FIGS. 1-4, the anti-roping elements 106 are each substantially straight (aka, substantially linear). In other embodiments, however, one or more the anti-roping elements 106 could foreseeably have different shapes, including for example, S-shaped, U-shaped, Zig-zag shaped, etc. Additionally, in FIGS. 1-4, each of the anti-roping elements 106 is a separate element. But, in other embodiments, any two or more of the elements 106 can be connected such that a single element 106 may extend the length of the suture 104, then include a U-shaped turn, and extend back along the length of the suture 104 adjacent to (e.g., parallel to) the preceding length. Also, in FIGS. 1-4, the anti-roping elements 106 are disposed parallel to each other and are equally spaced apart from each other. In alternative versions, the anti-roping elements 106 could have unequal spacing and/or could be disposed in a non-parallel manner. Further still, in FIGS. 1-4, the anti-roping elements 106 are depicted as having a thickness that is generally the same as the thickness of the other elements forming the mesh construct of the elongated suture 104. In other embodiments, any one or more of the anti-roping elements 106 could be thicker or thinner than the other elements forming the mesh construct of the elongated suture 104. Further yet, while FIGS. 1-4 show four (4) anti-roping elements, alternative embodiments could include any number so long as the desired objective is achieved without compromising or detracting from the macroporous character of the suture 104. Finally, while FIGS. 1-4 illustrate a hollow tubular suture 104, other embodiments of the medical device 100 as mentioned could include other geometries including, for example, a planar (e.g., flat ribbon) geometry. Therefore, it can be understood based on the foregoing description that the anti-roping elements 106 includes on such planar sutures 104 could include a plurality of substantially straight elements extending the length of the suture 104, and being parallel to each other and equally spaced apart. Alternatively, the anti-roping elements 106 on the planar suture 104 could take on any of the alternative constructs discussed with respect to the tubular construct expressly depicted in FIGS. 1-4.

Although the disclosure has been described in connection with specific preferred embodiments, it should be understood that the disclosure as claimed should not be unduly limited to such specific embodiments. Indeed, various modifications of the described modes for carrying out the disclosure would be apparent to those skilled in the relevant fields are intended to be within the scope of the present disclosure. For example, and importantly, although the application includes discrete descriptions of different embodiments of the invention, it can be understood that any features from one embodiment can be easily incorporated into any one or more of the other embodiments.

We claim:

1. A medical device comprising:
a surgical needle; and
an elongated suture having a first end attached to the surgical needle and a second end located away from the surgical needle, the elongated suture including a mesh wall and a plurality of pores extending through the mesh wall, at least some of the pores in the macroporous size range of greater than 200 microns and adapted to facilitate tissue integration through the mesh wall of the suture when introduced into a body,
wherein the suture is configured to resist a tensile load applied along an axial direction between the first and second ends of the elongated suture such that upon overloading the mesh wall collapses upon itself resulting in apparent suture elongation, collapsed diameter, and/or reduction in pore size.

2. The medical device of claim 1, further comprising one or more anti-roping elements fixed to the mesh wall, the anti-roping elements resisting the tensile load.

3. The medical device of claim 2, wherein the one or more anti-roping elements comprises one or more longitudinal fibers extending between the first and second ends of the elongated suture and either (a) fixed at one or more points to the mesh wall, or (b) not fixed to the mesh wall.

4. The medical device of claim 2, wherein the one or more anti-roping elements comprises a plurality of longitudinal elements fixed at a plurality of points to the mesh wall and extending between the first and second ends of the elongated suture.

5. The medical device of claim 4, wherein the plurality of longitudinal elements are parallel to each other and equally spaced from each other.

6. The medical device of claim 1, wherein the elongated suture comprises a hollow tubular mesh wall.

7. The medical device of claim 6, wherein the elongated suture has a diameter that is in a range of (a) approximately 1 mm to approximately 10 mm, or (b) approximately 1 mm to approximately 5 mm.

8. The medical device of claim 6, wherein the diameter of the suture is uniform along substantially the entire length of the suture between the first and second ends.

9. The medical device of claim 1, wherein the elongated suture comprises a planar mesh wall having a width dimension.

10. The medical device of claim 9, wherein the width dimension of the elongated suture is in a range of (a) approximately 1 mm to approximately 10 mm, or (b) approximately 1 mm to approximately 5 mm.

11. The medical device of claim 9, wherein the width dimension of the elongated suture is uniform along substantially the entire length of the suture between the first and second ends.

12. The medical device of claim 1, wherein the mesh wall of the suture extends along the entirety of the suture between the first and second ends.

13. The medical device of claim 1, having pores (a) greater than 200 microns to approximately 4 millimeters, (b) greater than 200 microns to approximately 2.5 millimeters, or (c) approximately 1 millimeter to approximately 2.5 millimeters.

14. The medical device of claim 1, wherein the plurality of pores vary in pore size.

15. The medical device of claim 1, wherein the suture is constructed of a material selected from the group consisting of: polyethylene terephthalate, nylon, polyolefin, polypropylene, silk, polymers p-dioxanone, co-polymer of p-dioxanone, ε-caprolactone, glycolide, L(−)-lactide, D(+)-lactide, meso-lactide, trimethylene carbonate, polydioxanone homopolymer, metal filaments, and combinations thereof.

16. The medical device of claim 1, wherein the suture is radially deformable such that the suture adopts a first cross-sectional profile in the absence of lateral stress and a second cross-sectional profile in the presence of lateral stress.

17. The medical device of claim 16, wherein the first cross-sectional profile exhibits radial symmetry.

18. The medical device of claim 16, wherein the second cross-sectional profile exhibits partially or wholly collapsed conformation.

19. The medical device of claim 1, wherein the suture has a circular cross-sectional profile when in a non-stressed state.

20. The medical device of claim 1, further comprising an anchor attached to the second end of the suture for preventing suture pull through during use, the anchor having a dimension that is larger than a diameter of the suture.

21. The medical device of claim 20, wherein the anchor comprises a loop, a ball, a disc, a cylinder, a barb, and/or a hook.

22. The medical device of claim 1, wherein the mesh wall comprises a woven or knitted mesh material.

23. The medical device of claim 1, where the elongated suture is greater than approximately 20 cm in length.

24. The medical device of claim 6, wherein the tubular mesh wall defines a hollow core.

25. The medical device of claim 24, wherein the hollow core defines a hollow cylindrical space devoid of suture material.

26. The medical device of clam 25, wherein the hollow core includes a honeycomb structure, a 3D lattice structure, or other suitable matrices defining one or more interior voids.

27. A method of re-apposing soft tissue, the method comprising:
piercing a portion of the soft tissue with a surgical needle attached to a first end of a mesh suture;
threading the mesh suture through the soft tissue, wherein the mesh suture comprises a mesh wall and a plurality of pores extending through the mesh wall, at least some of the pores having a pore size that is greater than or equal to approximately 200 microns such that the mesh suture is adapted to accommodate soft tissue growing through the mesh wall, thereby integrating with the suture; and
applying a tensile load to the mesh suture along an axial direction between the first and second ends, wherein the suture is configured to resist the tensile load such that upon overloading the mesh wall collapses upon itself resulting in apparent suture elongation, collapsed diameter, and/or reduction in pore size.

28. The method of claim 27, further comprising identifying that the suture is overloaded under tension by recognizing the elongation and/or reduction in pore size.

29. The method of claim 27, further comprising one or more anti-roping elements fixed to the mesh wall, the anti-roping elements resisting elongation while threading the mesh suture through the soft tissue and applying the tensile load.

30. The method of claim 27, wherein threading the tubular suture through the soft tissue comprises making a plurality of stitches.

31. The method of claim 27, further comprising anchoring the tubular suture in place in the soft tissue after threading the tubular suture through the soft tissue.

32. The method of claim 31, wherein anchoring the tubular suture in place comprises passing the surgical needle through a closed loop anchor at the second end of the tubular suture and creating a cinch for anchoring the suture to the soft tissue.
